(19) United States
(12) Patent Application Publication
(10) Pub. No.: US 2025/0145559 A1
(43) Pub. Date: May 8, 2025

(54) METHOD FOR REACTIVATING ALKANOLAMINE SOLUTION

(71) Applicant: PetroChina Company Limited, Beijing (CN)

(72) Inventors: Xiaoqin YAN, Beijing (CN); Li LIANG, Beijing (CN); Gang XIONG, Beijing (CN); Qingmei CHEN, Beijing (CN); Qisong LIU, Beijing (CN)

(21) Appl. No.: 19/013,571

(22) Filed: Jan. 8, 2025

Related U.S. Application Data
(63) Continuation of application No. PCT/CN2023/094570, filed on May 16, 2023.

(30) Foreign Application Priority Data
Jul. 12, 2022 (CN) 202210815702.X

(51) Int. Cl. C07C 213/10

(52) U.S. Cl. CPC C07C 213/10

(57) ABSTRACT

A method for reactivating alkanolamine solution, including the following steps: performing, by using a hydrogen phosphate ion remover, a first adsorption treatment on a to-be-reactivated alkanolamine solution to obtain a first solution and an adsorption system; performing, by successively using a hydroxide ion exchange resin and a methyldiethanolamine cation exchange resin, a second adsorption treatment on the first solution to obtain a second solution; performing a first mixing treatment on an ammonium sulfate solution and the adsorption system to obtain a third solution and a to-be-regenerated remover; performing a second mixing treatment on a hydrogen ion exchange resin and the third solution to obtain a fourth solution; and performing a third mixing treatment on the second solution and the fourth solution to obtain a reactivated alkanolamine solution.

### CROSS-REFERENCE TO RELATED APPLICATIONS

The present application is a continuation of International Application No. PCT/CN2023/094570, filed on May 16, 2023, which claims priority to Chinese Patent Application No. 202210815702.X, and filed with the China National Intellectual Property Administration on Jul. 12, 2022. The disclosures of the aforementioned applications are hereby incorporated by reference in their entireties.

### TECHNICAL FIELD

The embodiments of the present application relate to the technical field of chemical separation, and in particular, to a method for reactivating alkanolamine solution.

### BACKGROUND

The alkanolamine purification process refers to the process of desulfurization and decarbonization by using alkanolamine solution. The alkanolamine purification process is not only widely used in the purification of natural gas and refinery gas, but also frequently used in the industry of synthetic ammonia and the industry of preparing downstream products through synthesis gas.

In the prior art, amine hydrogen phosphate is usually added to an aqueous solution of N-methyldiethanolamine (MDEA) to reduce the pH of the aqueous solution of MDEA. The use of amine hydrogen phosphate and MDEA may produce a common ion effect to reduce the absorption rate of CO₂, thereby reducing the competitive absorption effect of CO₂ on H₂S and improving the selective absorption of H₂S. However, during the gas purification process, the complex alkanolamine solution formed by MDEA and amine hydrogen phosphate inevitably generates a variety of impurities due to autooxidation of the complex alkanolamine solution, or pollution of the complex alkanolamine solution by entrained substances in the feed gas. These impurities include heat-stable salts such as glycolate, acetate, formate, sulfate, chloride, oxalate, and thiosulfate. In the actual application process, heat-stable salts have strong corrosiveness, and may easily lead to safety problems such as corrosion perforation in alkanolamine purification devices. Furthermore, heat-stable salts may increase the viscosity of the complex alkanolamine solution after heat-stable salts accumulate to a certain concentration in the complex alkanolamine solution, and the mass transfer rate of acid gas in the complex alkanolamine solution is thus increased, resulting in a reduction of the desulfurization performance of the alkanolamine purification device and a production quality problem of excessive H₂S content in the product gas. At the same time, when the concentration of heat-stable salts in the complex alkanolamine solution further increases, some of heat-stable salts will precipitate and block the trays in the alkanolamine purification device, thereby causing the alkanolamine purification device to be difficult to operate. Therefore, in order to ensure the operation of the alkanolamine purification device safe and the quality of the product gas standard, it is necessary to remove heat-stable salts in the complex alkanolamine solution in time to reduce the concentration of heat-stable salts in the complex alkanolamine solution to be under a very low concentration level.

Existing methods for reactivating alkanolamine solution include vacuum distillation, electrodialysis and ion exchange. Regarding the vacuum distillation, although heat-stable salts can be separated by utilizing the characteristic that heat-stable salts have a high-boiling point, the amine hydrogen phosphate active agent is also easily separated in the process of the separation of heat-stable salts, because the amine hydrogen phosphate active agent used for enhancing the selective absorption of H₂S in the complex alkanolamine solution is also a substance having a high-boiling point. Regarding the electrodialysis method, although electric field can be used to allow anions of heat-stable salts in the complex alkanolamine solution to move towards the positive electrode and pass through the anion exchange membrane of the electrodialysis device, and allow cations of heat-stable salts in the complex alkanolamine solution to move towards the negative electrode and pass through the cation exchange membrane of the electrodialysis device, so as to achieve the purpose of removing anions and cations of heat-stable salts in the complex alkanolamine solution, amine hydrogen phosphate can also be decomposed into anions and cations in the electric field and removed. Regarding the ion exchange method, although anion exchange resins can be used to remove anions of heat-stable salts such as chloride ions, sulfate ions, thiosulfate ions, formate ions, acetate ions and oxalate ions in the complex alkanolamine solution to a low concentration level, hydrogen phosphate ions can also be removed. In summary, the existing methods for reactivating alkanolamine solution will not only remove heat-stable salts in the complex alkanolamine solution, but also remove the amine hydrogen phosphate active agent that enhances the selective absorption of H₂S, resulting in that the obtained reactivated alkanolamine solution has low selective absorption of H₂S, and thus it is difficult to obtain product gas with a qualified H₂S content.

Therefore, there is an urgent need of providing a method for reactivating alkanolamine solution, which is capable of removing heat-stable salts with almost no loss of amine hydrogen phosphate.

### SUMMARY

The present application provides a method for reactivating alkanolamine solution, which is capable of removing heat-stable salts in a to-be-reactivated alkanolamine solution with almost no loss of amine hydrogen phosphate in the to-be-reactivated alkanolamine solution.

The present application provides a method for reactivating alkanolamine solution, including the following steps:
performing, by using a hydrogen phosphate ion remover, a first adsorption treatment on a to-be-reactivated alkanolamine solution to obtain a first solution and an adsorption system;
performing, by successively using a hydroxide ion exchange resin and a methyldiethanolamine cation exchange resin, a second adsorption treatment on the first solution to obtain a second solution;
performing a first mixing treatment on an ammonium sulfate solution and the adsorption system to obtain a third solution and a to-be-regenerated remover; performing a second mixing treatment on a hydrogen ion exchange resin and the third solution to obtain a fourth solution;
performing a third mixing treatment on the second solution and the fourth solution to obtain a reactivated alkanolamine solution.

In the method for reactivating alkanolamine solution as described above, the hydrogen phosphate ion remover is [Ca₍₂₋₃₎Sc(OH)₍₆₋₈₎]OH.

In the method for reactivating alkanolamine solution as described above, a mass m₀ of the to-be-reactivated alkanolamine solution, a mass percentage p₀ of hydrogen phosphate ions in the to-be-reactivated alkanolamine solution, a mass m₁ of the hydrogen phosphate ion remover and an adsorption mass m₂ of per unit mass of the hydrogen phosphate ion remover satisfy the following relationship:

$${m}_{0} * {p}_{0} \leq {m}_{1} * {m}_{2} * 90 \% .$$

In the method for reactivating alkanolamine solution as described above, the to-be-reactivated alkanolamine solution is allowed to flow through the hydrogen phosphate ion remover to achieve the first adsorption treatment on the to-be-reactivated alkanolamine solution by the hydrogen phosphate ion remover;
a volume space velocity at which the to-be-reactivated alkanolamine solution flows through the hydrogen phosphate ion remover is less than or equal to 5 h⁻¹.

In the method for reactivating alkanolamine solution as described above, a volume V₁ of the ammonium sulfate solution, a volume molar concentration C₁ of the ammonium sulfate solution, a volume V₀ of the to-be-reactivated alkanolamine solution and a volume molar concentration C₀ of hydrogen phosphate ions in the to-be-reactivated alkanolamine solution satisfy the following relationship:

$${V}_{1} * {C}_{1} = 1.5 {V}_{0} * {C}_{0} ; and / or ,$$

the ammonium sulfate solution is allowed to flow through the adsorption system to achieve the first mixing treatment of the ammonium sulfate solution and the adsorption system;
a volume space velocity at which the ammonium sulfate solution flows through the adsorption system is less than or equal to 4 h⁻¹.

In the method for reactivating alkanolamine solution as described above, the ammonium sulfate solution has a volume molar concentration of 1.2-2.3 mol/L.

In the method for reactivating alkanolamine solution as described above, the method for reactivating alkanolamine solution further includes: performing, by using a regeneration liquid, a regeneration treatment on the to-be-regenerated remover to obtain a regenerated remover;
the regeneration liquid is selected from a sodium hydroxide aqueous solution and/or a potassium hydroxide aqueous solution.

In the method for reactivating alkanolamine solution as described above, hydroxyl ions in the regeneration liquid has a concentration of 1.3-1.5 mol/L.

In the method for reactivating alkanolamine solution as described above, a volume V₃ of the regeneration fluid and a volume V₂ of the to-be-regenerated remover satisfy the following relationship:

V₃≤V₂.

In the method for reactivating alkanolamine solution as described above, V₃=4V₂.

In the method for reactivating alkanolamine solution as described above, the regeneration liquid is allowed to flow through the to-be-regenerated remover to achieve the regeneration treatment on the to-be-regenerated remover by the regeneration liquid;
a volume space velocity at which the regeneration liquid flows through the to-be-regenerated remover is greater than or equal to 4 h⁻¹.

In the method for reactivating alkanolamine solution as described above, a volume space velocity at which the regeneration liquid flows through the to-be-regenerated remover is 6 h⁻¹.

By the method for reactivating alkanolamine solution of the present application, heat-stable salts in the to-be-reactivated alkanolamine solution can be removed with almost no loss of amine hydrogen phosphate. Since amine hydrogen phosphate has a strong selective absorption for H₂S, the reactivated alkanolamine solution obtained by the method for reactivating alkanolamine solution of the present application still has relatively strong selective absorption for H₂S. A product gas with qualified H₂S content can be obtained by alkanolamine purification using the reactivated alkanolamine solution of the present application.

### DESCRIPTION OF EMBODIMENTS

In order to make the purpose, technical solutions and advantages of the present application clearer, the technical solutions in the embodiments of the present application will be clearly and completely described below. Apparently, the described embodiments are some rather than all embodiments of the present application. Based on the embodiments in the present application, all other embodiments obtained by persons of ordinary skill in the art without creative work fall within the protection scope of the present application.

The present application provides a method for reactivating alkanolamine solution, including the following steps:
S101: performing, by using a hydrogen phosphate ion remover, a first adsorption treatment on a to-be-reactivated alkanolamine solution to obtain a first solution and an adsorption system;
S102: performing, by successively using a hydroxide ion exchange resin and a methyldiethanolamine cation exchange resin, a second adsorption treatment on the first solution to obtain a second solution;
S103: performing a first mixing treatment on an ammonium sulfate solution and the adsorption system to obtain a third solution and a to-be-regenerated remover;
S104: performing a second mixing treatment on a hydrogen ion exchange resin and the third solution to obtain a fourth solution;
S105: performing a third mixing treatment on the second solution and the fourth solution to obtain a reactivated alkanolamine solution.

Specifically, in S101, the to-be-reactivated alkanolamine solution is a solution, which contains heat-stable salts, amine hydrogen phosphate, and N-methyldiethanolamine (MDEA) and is produced in an alkanolamine purification process. Heat-stable salts include at least one of chloride, sulfate, thiosulfate, formate, acetate, glycolate or oxalate. Based on a total mass of the to-be-reactivated alkanolamine solution, the content of heat-stable salts is 10 ppm-100,000 ppm, and the mass percentage of MDEA is 30%-50%. The hydrogen phosphate ion remover is any material with extremely high selective absorption for hydrogen phosphate ions. The hydrogen phosphate ion remover is composed of anions and cations. Due to the interaction force between cations in the hydrogen phosphate ion remover and hydrogen phosphate ions is greater than the interaction force between cations and anions in the hydrogen phosphate ion remover, anions in the hydrogen phosphate ion remover is easily substituted by hydrogen phosphate ions, thereby achieving that hydrogen phosphate ions are adsorbed to the hydrogen phosphate ion remover. In The present application, by using a hydrogen phosphate ion remover with extremely high selective absorption for hydrogen phosphate ions, the first adsorption treatment on the to-be-reactivated alkanolamine solution containing heat-stable salts and amine hydrogen phosphate is achieved. During the first adsorption treatment process, anions in the hydrogen phosphate ion remover exchange with hydrogen phosphate ions in the to-be-reactivated alkanolamine solution, and hydrogen phosphate ions in the to-be-reactivated alkanolamine solution are adsorbed to the hydrogen phosphate ion remover, and thus a first solution in which hydrogen phosphate ions are removed is formed, and an adsorption system to which hydrogen phosphate ions are adsorbed is formed.

In S102, the second adsorption treatment includes the following. An adsorption treatment is performed on the first solution formed in S101 by using a hydroxide ion exchange resin. Anions of heat-stable salts in the first solution exchange with hydroxide ions in the hydroxide ion exchange resin, heat-stable salts are converted into inorganic alkalis, and thus a solution containing inorganic alkalis is formed. Then an adsorption treatment is performed on the solution containing inorganic alkalis by using a methyldiethanolamine cation exchange resin. Cations in the inorganic alkalis exchange with methyldiethanolamine cations in the methyldiethanolamine cation exchange resin, and thus the inorganic alkalis are converted into water and methyldiethanolamine, and thus a second solution containing water and methyldiethanolamine is obtained.

In S103, an ammonium sulfate solution and the adsorption system obtained in S101 are mixed for a first mixing treatment. On the one hand, the ammonium sulfate solution has a pH of 4-5, which is weakly acidic and does not easily destroy the crystal structure of the adsorption system. On the other hand, in an environment with a pH of 4-5, phosphate ions in the adsorption system are converted into dihydrogen phosphate ions, and thus an adsorption system containing dihydrogen phosphate ions is obtained. The interaction force between dihydrogen phosphate ions and cations in the adsorption system is much smaller than the interaction force between sulfate ions and cations in the adsorption system. Therefore, dihydrogen phosphate ions in the adsorption system exchange with sulfate ions to remove dihydrogen phosphate ions in the adsorption system, and thus a third solution containing dihydrogen phosphate is obtained. At the same time, the adsorption system is subjected to the first mixing treatment to obtain a to-be-regenerated remover containing sulfate ions.

In S104, a hydrogen ion exchange resin and the third solution containing dihydrogen phosphate are subjected to a second mixing treatment. In the second mixing treatment, cations of the dihydrogen phosphate exchange with hydrogen ions in the hydrogen ion exchange resin, and thus dihydrogen phosphate is converted into phosphoric acid, and a fourth solution containing phosphoric acid is obtained.

In S105, the second solution obtained in S102 and the fourth solution obtained in S104 are subjected to a third mixing treatment. During the third mixing treatment, phosphoric acid in the fourth solution react with N-methyldiethanolamine (MDEA) in the second solution to obtain amine hydrogen phosphate, and thus a reactivated alkanolamine solution, in which amine hydrogen phosphate is contained and heat-stable salts are removed, can be obtained.

It can be understood that the specific method of the first adsorption treatment is not particularly limited in the present application, as long as the hydrogen phosphate ion remover can adsorb hydrogen phosphate ions in the to-be-reactivated alkanolamine solution. For example, the first adsorption treatment may be achieved by directly mixing the hydrogen phosphate ion remover and the to-be-reactivated alkanolamine solution, where the hydrogen phosphate ion remover is used to adsorb hydrogen phosphate ions in the to-be-reactivated alkanolamine solution. The first adsorption treatment may also be achieved by filling the hydrogen phosphate ion remover into a glass column and introducing the to-be-reactivated alkanolamine solution into the glass column filled with the hydrogen phosphate ion remover.

The specific method of the second adsorption treatment is not particularly limited in the present application, as long as heat-stable salts in the first solution can be converted into methyldiethanolamine and water. For example, the second adsorption treatment may be achieved by mixing the first solution with a hydroxide ion exchange resin to obtain a solution containing inorganic alkalis and then mixing the solution containing inorganic alkalis with a methyldiethanolamine cation exchange resin to obtain a second solution containing water and methyldiethanolamine. The second adsorption treatment may also be achieved by filling glass columns with a hydroxide ion exchange resin and a methyldiethanolamine cation exchange resin respectively, and allowing the first solution to flow through the glass column filled with the hydroxide ion exchange resin and the glass column filled with the methyldiethanolamine cation exchange resin successively. The second adsorption treatment may also be achieved by filling only a hydroxide ion exchange resin into a glass column, allowing the first solution to flow through the glass column filled with hydroxide ion exchange resin to obtain a solution containing inorganic alkalis, and then mixing the solution containing inorganic alkalis with a methyldiethanolamine cation exchange resin. The second adsorption treatment may also be achieved by filling only a methyldiethanolamine cation exchange resin into a glass column, mixing the first solution with a hydroxide ion exchange resin to obtain a solution containing inorganic alkalis, and then introducing the solution containing inorganic alkalis into the glass column filled with the methyldiethanolamine cation exchange resin.

The specific methods of the first mixing treatment and the second mixing treatment are not particularly limited in the present application, as long as the first mixing treatment and the second mixing treatment can be achieved. Specifically, the methods of the first mixing treatment and the second mixing treatment may refer to the first mixing treatment.

The hydroxide ion exchange resin, the methyldiethanolamine cation exchange resin and the hydrogen ion exchange resin are not particularly limited in the present application. Commonly used hydroxide ion exchange resins, methyldiethanolamine cation exchange resins and hydrogen ion exchange resins in the art may be selected to use.

The ammonium sulfate solution is not particularly limited in the present application. Commonly used ammonium sulfate solutions in the art may be selected to use. For example, an ammonium sulfate aqueous solution may be selected to use.

The sequential order of S102 and S103 is not limited in the present application. In an actual application process, S102 may be performed first, or S103 may also be performed first, or S102 and S103 may be performed simultaneously.

For the method for reactivating alkanolamine solution of the present application, the removal rate of heat-stable salts may be greater than 96% and the retention rate of amine hydrogen phosphate may be greater than 98.5% (the removal rate of amine hydrogen phosphate is less than or equal to 1.5%). Compared with the existing methods for reactivating alkanolamine solution (the removal rate of heat-stable salts is greater than 94% and the retention rate of amine hydrogen phosphate is less than 2%), the method for reactivating alkanolamine solution in the present application has excellent removal rate of heat-stable salts and excellent retention rate of amine hydrogen phosphate. Therefore, the reactivated alkanolamine solution obtained by the method for reactivating alkanolamine solution of the present application has relatively excellent selective absorption for H₂S, and this is beneficial to obtaining a product gas with H₂S content qualified. Moreover, the reactivated alkanolamine solution has low corrosiveness, and this is beneficial to avoiding corrosion of the alkanolamine purification device, so that the alkanolamine purification device can operate stably for a long time.

It is worth mentioning that by using the method for reactivating alkanolamine solution in the present application, the alkanolamine solution can be recycled and reused, thereby effectively reducing the production cost and environmental protection pressure of factories, and obtaining relatively excellent economic and social benefits.

In some embodiments of the present application, the hydrogen phosphate ion remover is [Ca₍₂₋₃₎Sc(OH)₍₆₋₈₎]OH.

[Ca₍₂₋₃₎Sc(OH)₍₆₋₈₎]OH has extremely high selective absorption for hydrogen phosphate ions. Specifically, a ratio of the selective absorption of [Ca₍₂₋₃₎Sc(OH)₍₆₋₈₎]OH for hydrogen phosphate ions to the selective absorption of [Ca₍₂₋₃₎Sc(OH)₍₆₋₈₎]OH for anions of heat-stable salts is greater than 6000; and the adsorption capacity of [Ca₍₂₋₃₎Sc(OH)₍₆₋₈₎]OH for hydrogen phosphate ions (the adsorption mass of per unit mass of [Ca₍₂₋₃₎Sc(OH)₍₆₋₈₎]OH) is greater than 169 mg/g. Therefore, the selection of [Ca₍₂₋₃₎Sc(OH)₍₆₋₈₎]OH as the hydrogen phosphate ion remover not only can achieve the selective adsorption of hydrogen phosphate ions in the to-be-reactivated alkanolamine solution to a great extent, but also can achieve the adsorption of a large amount of hydrogen phosphate ions, thereby facilitating industrial applications.

In some embodiments of the present application, in order to achieve sufficient adsorption of hydrogen phosphate ions in the to-be-reactivated alkanolamine solution by the hydrogen phosphate ion remover, the mass m₀ of the to-be-reactivated alkanolamine solution, the mass percentage p₀ of hydrogen phosphate ions in the to-be-reactivated alkanolamine solution, the mass m₁ of the hydrogen phosphate ion remover, and the adsorption mass m₂ of per unit mass of the hydrogen phosphate ion remover are specifically selected. Such that the mass m₀ of the to-be-reactivated alkanolamine solution, the mass percentage p₀ of the hydrogen phosphate ions in the to-be-reactivated alkanolamine solution, the mass m₁ of the hydrogen phosphate ion remover and the adsorption mass m₂ of per unit mass of the hydrogen phosphate ion remover satisfy the following relationship:

$${m}_{0} * {p}_{0} \leq {m}_{1} * {m}_{2} * 90 \% .$$

In some embodiments of the present application, in order to make the to-be-reactivated alkanolamine solution fully contact with the hydrogen phosphate ion remover to make the hydrogen phosphate ion remover adsorb as much hydrogen phosphate ions as possible in the to-be-reactivated alkanolamine solution, the to-be-reactivated alkanolamine solution may flow through the hydrogen phosphate ion remover to achieve the first adsorption treatment on the to-be-reactivated alkanolamine solution by the hydrogen phosphate ion remover.

The volume space velocity at which the to-be-reactivated alkanolamine solution flows through the hydrogen phosphate ion remover is less than or equal to 5 h⁻¹.

In a specific embodiment, the volume space velocity at which the to-be-reactivated alkanolamine solution flows through the hydrogen phosphate ion remover is 4 h⁻¹.

In the present application, in order to obtain dihydrogen phosphate ions as much as possible from the adsorption system and thus to obtain hydrogen phosphate ions as much as possible, the volume V₁ of the ammonium sulfate solution, the volume molar concentration C₁ of the ammonium sulfate solution, the volume V₀ of the to-be-reactivated alkanolamine solution, and the volume molar concentration C₀ of hydrogen phosphate ions in the to-be-reactivated alkanolamine solution are specifically selected.

For example, the volume V₁ of the ammonium sulfate solution, the volume molar concentration C₁ of the ammonium sulfate solution, the volume V₀ of the to-be-reactivated alkanolamine solution, and the volume molar concentration C₀ of hydrogen phosphate ions in the to-be-reactivated alkanolamine solution satisfy the following relationship:

$${V}_{1} * {C}_{1} = 1.5 {V}_{0} * {C}_{0} .$$

In some embodiments of the present application, in order to make the ammonium sulfate solution fully contact with the adsorption system to obtain dihydrogen phosphate ions as much as possible from the adsorption system, and thus to obtain hydrogen phosphate ions as much as possible, the ammonium sulfate solution may flow through the adsorption system to achieve the first mixing treatment of the ammonium sulfate solution and the adsorption system.

Further, the volume space velocity at which the ammonium sulfate solution flows through the adsorption system is less than or equal to 4 h⁻¹.

In a specific embodiment, the volume space velocity at which the ammonium sulfate solution flows through the adsorption system is 3 h⁻¹.

In some embodiments of the present application, the ammonium sulfate solution has a volume molar concentration of 1.2-2.3 mol/L.

In the present application, when the molar concentration of the ammonium sulfate solution conforms the above range, the ammonium sulfate solution has a more suitable pH, and dihydrogen phosphate ions can be obtained from the adsorption system without destroying the crystal structure of the adsorption system, and thus phosphate ions are obtained. In addition, when the molar concentration of the ammonium sulfate solution conforms the above range, the ammonium sulfate solution has an appropriate volume, and can completely infiltrate the adsorption system, and is helpful to completely remove hydrogen phosphate ions in the adsorption system. Further, the molar concentration of the ammonium sulfate solution is 1.9-2.0 mol/L.

In some embodiments of the present application, the method for reactivating alkanolamine solution further includes: perform, by using a regeneration liquid, a regeneration treatment on the to-be-regenerated remover to obtain a regenerated remover;
the regeneration liquid is selected from a sodium hydroxide aqueous solution and/or a potassium hydroxide aqueous solution.

In the present application, the ammonium sulfate solution and the adsorption system are subjected to a first mixing treatment to obtain a to-be-regenerated remover containing sulfate ions. The to-be-regenerated remover containing sulfate ions is subjected to a regeneration treatment by using a sodium hydroxide aqueous solution and/or a potassium hydroxide aqueous solution. Hydroxide ions in the sodium hydroxide aqueous solution and/or the potassium hydroxide aqueous solution exchange with sulfate ions in the to-be-regenerated remover to form a regenerated remover.

The specific method of the regeneration treatment is not limited in the present application, as long as hydroxide ions in the sodium hydroxide aqueous solution and/or potassium hydroxide aqueous solution exchange with sulfate ions in the to-be-regenerated remover to form the regenerated remover. For example, the regeneration treatment may be achieved by static immersion of the to-be-regenerated remover into a sodium hydroxide aqueous solution and/or a potassium hydroxide aqueous solution; the regeneration treatment may also be achieved by placing the to-be-regenerated remover into a glass column and allowing a regeneration liquid to flow through the glass column filled with the to-be-regenerated remover.

The flow rate of the regeneration liquid for passing through the glass column filled with the to-be-regenerated remover is not limited in the present application. In some embodiments, when the flow rate of the regeneration liquid for passing through the glass column filled with the to-be-regenerated remover is larger, it is conducive to making a liquid layer at the interface between the to-be-regenerated remover and the regeneration liquid has a higher concentration of hydroxide ions and a lower concentration of sulfate ions. This is conducive to the diffusion of hydroxide ions in the regeneration liquid into the inside of the to-be-regenerated remover, so that sulfate ions in the to-be-regenerated remover enter the regeneration liquid. The full regeneration of the to-be-regenerated remover is thus achieved, thereby obtaining a regenerated remover with excellent quality.

Further, in some embodiments of the present application, the regeneration treatment on the to-be-regenerated remover by the regeneration liquid is achieved by allowing the regeneration liquid to flow through the to-be-regenerated remover;
the volume space velocity at which the regeneration liquid flows through the to-be-regenerated remover is greater than or equal to 4 h⁻¹.

In the present application, the use of the regeneration liquid to perform the regeneration treatment on the to-be-regenerated remover can obtain a regenerated remover with excellent quality, and achieve the regeneration and reuse of the hydrogen phosphate ion remover, and this has excellent economic value.

In some embodiments of the present application, in order to make the regeneration liquid fully infiltrate the to-be-regenerated remover, to achieve complete removal of sulfate ions in the to-be-regenerated remover and to obtain a regenerated remover with excellent quality, the concentration of hydroxide ions in the regeneration liquid is 1.3-1.5 mol/L.

In some embodiments of the present application, in order to remove as much phosphate ions as possible from the to-be-regenerated remover and obtain a regenerated remover with excellent quality, the volume V₃ of the regeneration liquid and the volume V₂ of the to-be-regenerated remover V₂ can be specifically selected. For example, the volume V₃ of the regeneration fluid and the volume V₂ of the to-be-regenerated remover satisfy the following relationship:

V₃≥3V₂.

In a specific embodiment, the volume V₃ of the regeneration liquid and the volume V₂ of the to-be-regenerated remover satisfy V₃=4V₂, and the volume space velocity at which the regeneration liquid flows through the to-be-regenerated remover is 6 h⁻¹.

In the following, the technical solutions of the present application will be further described with reference to specific embodiments.

### Example 1

A method for reactivating alkanolamine solution in the present example includes the following steps.

### 1) To-be-Reactivated Alkanolamine Solution

Based on a total mass of a to-be-reactivated alkanolamine solution, the mass percentage of heat-stable salts was 1.4%, the mass percentage of N-methyldiethanolamine was 45%, the mass percentage of hydrogen phosphate ions was 4%, and the volume concentration of hydrogen phosphate ions is 0.43 mol/L.

In the to-be-reactivated alkanolamine solution, the mass ratio of sulfate ions, thiosulfate ions, chloride ions, formate ions, acetate ions, glycolate ions, and oxalate ions is 1:1:1:1:1:1:1.

### 2) Processing Column

Column No. 1: A glass column with an inner diameter of 25 mm is filled with 100 g of a hydrogen phosphate ion remover [Ca₃Sc(OH)₈]OH, and the adsorption capacity of [Ca₃Sc(OH)₈]OH is 174 mg/g.

Column No. 2: A glass column with an inner diameter of 25 mm is filled with 100 g of a hydroxide ion exchange resin.

Column No. 3: A glass column with an inner diameter of 25 mm is filled with 100 g of a methyldiethanolamine ion exchange resin.

Column No. 4: A glass column with an inner diameter of 25 mm is filled with 100 g of a hydrogen ion exchange resin.

### 3) First Reactivation Treatment

The to-be-reactivated alkanolamine solution flows through Column No. 1 for a first adsorption treatment, and the solution retained in Column No. 1 is discharged from top to bottom by using nitrogen to obtain a first solution, and the hydrogen phosphate ion remover is converted into an adsorption system.

The first solution flows through Column No. 2 and Column No. 3 in sequence for a second adsorption treatment, and the solutions retained in Column No. 2 and Column No. 3 are discharged from top to bottom by using nitrogen to obtain a second solution.

An ammonium sulfate solution flows through Column No. 1 filled with the adsorption system for a first mixing treatment, and the solution retained in Column No. 1 is discharged from top to bottom by using nitrogen to obtain a third solution, and the adsorption system in Column No. 1 is converted into a to-be-reactivated remover.

The third solution flows through Column No. 4 for a second mixing treatment, and the solution retained in Column No. 4 is discharged from top to bottom by using nitrogen to obtain a fourth solution.

The fourth solution is mixed with the second solution for a third mixing treatment to obtain a first reactivated alkanolamine solution.

Where, the to-be-reactivated alkanolamine solution has a mass of 380 g, and has a volume of 365 mL. The volume space velocity at which the to-be-reactivated alkanolamine solution flows through Column No. 1 is 5 h⁻¹; and the volume space velocity at which the first solution flows through Column No. 2 and Column No. 3 is 5 h⁻¹, respectively, and the to-be-regenerated remover has a volume of 125 mL.

The ammonium sulfate solution is an ammonium sulfate aqueous solution, and the ammonium sulfate aqueous solution has a volume molar concentration of 1.2 mol/L, and has a volume of 197 mL. The volume space velocity at which the ammonium sulfate aqueous solution flows through Column No. 1 is 4 h⁻¹. The volume space velocity at which the third solution flows through Column No. 4 is 4 h⁻¹.

4) Regeneration Treatment of the to-be-Regenerated Remover

A regeneration liquid flows through Column No. 1, then distilled water flows through Column No. 1, and finally the solution retained in Column No. 1 is discharged from top to bottom by using nitrogen, and thus the to-be-regenerated remover in Column No. 1 is regenerated to form a regenerated remover.

Where, the regeneration liquid is a sodium hydroxide aqueous solution, the molar concentration of hydroxide ions in the regeneration liquid is 1.3 mol/L, the regeneration liquid has a volume of 500 mL, and the volume space velocity at which the regeneration liquid flows through Column No. 1 is 5 h⁻¹.

The distilled water has a volume of 120 mL, and the volume space velocity at which the distilled water flows through Column No. 1 is 4 h⁻¹.

### 5) Regeneration Treatment of Column No. 2, Column No. 3 and Column No. 4

400 mL of a sodium hydroxide aqueous solution with a mass fraction of 4% flows through Column No. 2, and the solution retained in Column No. 2 is discharged from top to bottom by using nitrogen, and thus the resin in Column No. 2 is regenerated.

400 mL of a methyldiolamine chloride aqueous solution with a mass fraction of 3% flows through Column No. 3, and the solution retained in Column No. 3 is discharged from top to bottom by using nitrogen, and thus the resin in Column No. 3 is regenerated.

300 mL of a hydrochloric acid aqueous solution with a mass fraction of 3% flows through Column No. 4, and the solution retained in Column No. 4 is discharged from top to bottom by using nitrogen, and thus the resin in Column No. 4 is regenerated.

### 6) Second Reactivation Treatment

Step 3) is repeated to obtain a second reactivated alkanolamine solution.

7) The mass and volume of the first reactivated alkanolamine solution and of the second reactivated alkanolamine solution are measured. The concentration of amine hydrogen phosphate and the concentrations of various anions of the heat-stable salts in the first reactivated alkanolamine solution and the second reactivated alkanolamine solution are determined. The removal rates of amine hydrogen phosphate as well as various anions of the heat-stable salts in the first reactivated alkanolamine solution and the second reactivated alkanolamine solution are calculated, and the results are shown in Table 1.

TABLE 1

| | Removal rate/% | |
|---|---|---|
| | First reactivated | Second reactivated |
| Ingredient | alkanolamine solution | alkanolamine solution |
| Glycolate ion | 97.1 | 97.0 |
| Acetate ion | 97.4 | 97.4 |
| Formate ion | 97.8 | 97.6 |
| Chloride ion | 97.4 | 97.5 |
| Sulfate ion | 97.0 | 97.0 |
| Oxalate ion | 96.4 | 96.2 |
| Thiosulfate ion | 96.2 | 96.1 |
| Amine hydrogen | 1.3 | 1.5 |
| phosphate | | |

As can be seen from Table 1, in the method for reactivating alkanolamine solution of the present application, the removal rate of heat-stable salts is >96%, and the removal rate of amine hydrogen phosphate is <2.0%, indicating that by using the method for reactivating alkanolamine solution of the present application, heat-stable salts can be removed, and at the same time, amine hydrogen phosphate in the to-be-reactivated alkanolamine solution can be retained.

In addition, since the removal rate of amine hydrogen phosphate is less than 2.0% in both the first reactivated alkanolamine solution and the second reactivated alkanolamine solution, it indicates that the method for reactivating alkanolamine solution of the present application allows the hydrogen phosphate ion remover to be regenerated and reused repeatedly.

### Example 2

The method for reactivating alkanolamine solution of the present example is basically the same as that in Example 1, while the difference lies in that the hydrogen phosphate ion remover in Step 2) is ZnAl layered bimetallic hydroxide.

### Example 3

The method for reactivating alkanolamine solution of the present example is basically the same as that in Example 1, while the differences lie in:
in step 3), the volume space velocity at which the to-be-reactivated alkanolamine solution flows through Column No. 1 is 6 h⁻¹, and the volume space velocity at which the first solution flows through Column No. 2 and Column No. 3 is 5 h⁻¹, respectively;
the volume space velocity at which the ammonium sulfate aqueous solution flows through Column No. 1 is 5 h⁻¹; and
in step 4), the volume space velocity at which the regeneration liquid flows through Column No. 1 is 3 h⁻¹.

### Comparative Examples

The to-be-reactivated alkanolamine solution in Example 1 is reactivated by using an ion exchange method, an electrodialysis method, and a vacuum distillation method to obtain a reactivated alkanolamine solution respectively.

Specifically, the ion exchange method: the to-be-reactivated alkanolamine solution flows through an anion exchange resin from top to bottom, and the solution flowed out is the reactivated alkanolamine solution;
where, the volume of the anion exchange resin is 100 mL, and the volume of the to-be-reactivated alkanolamine solution is 150 mL;
the volume space velocity at which the to-be-reactivated alkanolamine solution flows through the anion exchange resin is 4 h⁻¹.

The electrodialysis method: a clamping device is used to clamp components, i.e. anion and cation exchange membranes, concentrated and fresh water spacer plates, positive and negative electrodes, an electrode frame, and a water guide plate, so as to form an electrodialysis device. 25 g of sodium hydroxide is added into 500 g of the to-be-reactivated alkanolamine solution, and the solution is mixed to fully react for 10 minutes. The sodium hydroxide reacts with heat-stable salts to form heat-stable sodium salts and MDEA, and the reacted solution is poured into the electrodialysis device and energized with direct current. Since heat-stable sodium salts are strong electrolytes, they are ionized into positively charged sodium ions and negatively charged anions. Under the action of the direct current electric field, sodium ions pass through the cation exchange membrane and anions pass through the anion exchange membrane, so as to enter the concentrated salt water tank. While MDEA and water are weak electrolytes and hardly ionized, they remain in the fresh water tank. The solution left in the fresh water tank is the reactivated alkanolamine solution.

The vacuum distillation method: 200 g of a to-be-reactivated alkanolamine solution is added into a distillation bottle, and the distillation system is vacuumized until the pressure therein is 90.3 kPa. The solution is heated to 150° C., and distilled for 20 minutes, and thus the solution in a receiving bottle is the reactivated alkanolamine solution.

The concentrations of amine hydrogen phosphate and heat-stable salts in the reactivated alkanolamine solutions obtained by the ion exchange method, the electrodialysis method, and the vacuum distillation method are measured respectively. The removal rates of amine hydrogen phosphate and heat-stable salts in the reactivated alkanolamine obtained by the ion exchange method, the electrodialysis method, and the vacuum distillation method are calculated respectively, and the results are shown in Table 2.

The concentrations of heat-stable salts in the second reactivated alkanolamine solutions in Examples 1-3 are directly measured using ion chromatography SY/T 7001. The removal rates of heat-stable salts in the second reactivated alkanolamine solutions are calculated according to the following formula. The results are shown in Table 2.

Removal rate of heat-stable salts=(total mass fraction of anions of heat-stable salts before reactivation−total mass fraction of anions of heat-stable salts after reactivation)÷total mass fraction of anions of heat-stable salts before reactivation×100%.

TABLE 2

| | Removal rate/% | | | | | |
|---|---|---|---|---|---|---|
| | Ion | | Vacuum | | | |
| | exchange | Electrodialysis | distillation | Example | Example | Example |
| Ingredient | method | method | method | 1 | 2 | 3 |
| Heat-stable | 94.7 | 95.3 | 98.4 | 96.8 | 69.4 | 88 |
| salts | | | | | | |
| Amine | 98.1 | 98.0 | 98.2 | 1.5 | 29.0 | 10.3 |
| hydrogen | | | | | | |
| phosphate | | | | | | |

As can be seen from Table 2, when a to-be-reactivated alkanolamine solution is treated by using the method for reactivating alkanolamine solution of the present application, not only most of heat-stable salts in the to-be-reactivated alkanolamine solution are removed, but also almost no amine hydrogen phosphate in the to-be-reactivated alkanolamine solution is lost.

In particular, by using the hydrogen phosphate ion remover [Ca₍₂₋₃₎Sc(OH)₍₆₋₈₎]OH, and controlling the volume space velocity at which the to-be-reactivated alkanolamine solution flows through the hydrogen phosphate ion remover, the volume space velocity at which the ammonium sulfate solution flows through the adsorption system, and the volume space velocity at which the regeneration liquid flows through the to-be-regenerated remover, heat-stable salts in the to-be-reactivated alkanolamine solution can be better removed, and almost no amine hydrogen phosphate in the to-be-reactivated alkanolamine solution is lost. In the method for reactivating alkanolamine solution of the present application, amine hydrogen phosphate can be better retained in the to-be-reactivated alkanolamine solution, and at the same time, more heat-stable salts in the to-be-reactivated alkanolamine solution can be removed (the removal rate of heat-stable salts is >96%, the removal rate of amine hydrogen phosphate is 1.5%, and the retention rate of amine hydrogen phosphate is 98.5%), compared with the ion exchange method, electrodialysis method, and vacuum distillation method (the removal rate of heat-stable salts is greater than 94%, the removal rate of amine hydrogen phosphate is greater than 98%, and the retention rate of amine hydrogen phosphate is less than 2%).

Finally, it should be noted that the above various embodiments are only merely intended for describing the technical solutions of the present application other than limiting the present application. Although the present application has been described in detail with reference to the aforementioned embodiments, persons of ordinary skill in the art should understand that they may still make modifications to the technical solutions described in the foregoing embodiments or make equivalent substitutions to some or all technical features thereof. These modifications or substitutions do not make the essence of the corresponding technical solutions depart from the scope of the technical solutions of embodiments of the present application.

What is claimed is:

1. A method for reactivating alkanolamine solution, comprising the following steps:
performing, by using a hydrogen phosphate ion remover, a first adsorption treatment on a to-be-reactivated alkanolamine solution to obtain a first solution and an adsorption system;
performing, by successively using a hydroxide ion exchange resin and a methyldiethanolamine cation exchange resin, a second adsorption treatment on the first solution to obtain a second solution;
performing a first mixing treatment on an ammonium sulfate solution and the adsorption system to obtain a third solution and a to-be-regenerated remover;
performing a second mixing treatment on a hydrogen ion exchange resin and the third solution to obtain a fourth solution; and
performing a third mixing treatment on the second solution and the fourth solution to obtain a reactivated alkanolamine solution.

2. The method for reactivating alkanolamine solution according to claim 1, wherein the hydrogen phosphate ion remover is [Ca₍₂₋₃₎Sc(OH)₍₆₋₈₎]OH.

3. The method for reactivating alkanolamine solution according to claim 1, wherein a mass m₀ of the to-be-reactivated alkanolamine solution, a mass percentage p₀ of hydrogen phosphate ions in the to-be-reactivated alkanolamine solution, a mass m₁ of the hydrogen phosphate ion remover, and an adsorption mass m₂ of per unit mass of the hydrogen phosphate ion remover satisfy the following relationship:
$${m}_{0} * {p}_{0} \leq {m}_{1} * {m}_{2} * 90 \% .$$

4. The method for reactivating alkanolamine solution according to claim 2, wherein a mass m₀ of the to-be-reactivated alkanolamine solution, a mass percentage p₀ of hydrogen phosphate ions in the to-be-reactivated alkanolamine solution, a mass m₁ of the hydrogen phosphate ion remover, and an adsorption mass m₂ of per unit mass of the hydrogen phosphate ion remover satisfy the following relationship:
$${m}_{0} * {p}_{0} \leq {m}_{1} * {m}_{2} * 90 \% .$$

5. The method for reactivating alkanolamine solution according to claim 1, comprising allowing the to-be-reactivated alkanolamine solution to flow through the hydrogen phosphate ion remover to achieve the first adsorption treatment on the to-be-reactivated alkanolamine solution by the hydrogen phosphate ion remover;
a volume space velocity at which the to-be-reactivated alkanolamine solution flows through the hydrogen phosphate ion remover is less than or equal to 5 h⁻¹.

6. The method for reactivating alkanolamine solution according to claim 2, comprising allowing the to-be-reactivated alkanolamine solution to flow through the hydrogen phosphate ion remover to achieve the first adsorption treatment on the to-be-reactivated alkanolamine solution by the hydrogen phosphate ion remover;
a volume space velocity at which the to-be-reactivated alkanolamine solution flows through the hydrogen phosphate ion remover is less than or equal to 5 h⁻¹.

7. The method for reactivating alkanolamine solution according to claim 3, comprising allowing the to-be-reactivated alkanolamine solution to flow through the hydrogen phosphate ion remover to achieve the first adsorption treatment on the to-be-reactivated alkanolamine solution by the hydrogen phosphate ion remover;
a volume space velocity at which the to-be-reactivated alkanolamine solution flows through the hydrogen phosphate ion remover is less than or equal to 5 h⁻¹.

8. The method for reactivating alkanolamine solution according to claim 4, comprising allowing the to-be-reactivated alkanolamine solution to flow through the hydrogen phosphate ion remover to achieve the first adsorption treatment on the to-be-reactivated alkanolamine solution by the hydrogen phosphate ion remover;
a volume space velocity at which the to-be-reactivated alkanolamine solution flows through the hydrogen phosphate ion remover is less than or equal to 5 h⁻¹.

9. The method for reactivating alkanolamine solution according to claim 1, wherein a volume V₁ of the ammonium sulfate solution, a volume molar concentration C₁ of the ammonium sulfate solution, a volume V₀ of the to-be-reactivated alkanolamine solution, and a volume molar concentration C₀ of hydrogen phosphate ions in the to-be-reactivated alkanolamine solution satisfy the following relationship:
$${V}_{1} * {C}_{1} = 1.5 {V}_{0} * {C}_{0} ; and / or ,$$
the ammonium sulfate solution is allowed to flow through the adsorption system to achieve the first mixing treatment of the ammonium sulfate solution and the adsorption system;
a volume space velocity at which the ammonium sulfate solution flows through the adsorption system is less than or equal to 4 h⁻¹.

10. The method for reactivating alkanolamine solution according to claim 2, wherein a volume V₁ of the ammonium sulfate solution, a volume molar concentration C₁ of the ammonium sulfate solution, a volume V₀ of the to-be-reactivated alkanolamine solution, and a volume molar concentration C₀ of hydrogen phosphate ions in the to-be-reactivated alkanolamine solution satisfy the following relationship:
$${V}_{1} * {C}_{1} = 1.5 {V}_{0} * {C}_{0} ; and / or ,$$
the ammonium sulfate solution is allowed to flow through the adsorption system to achieve the first mixing treatment of the ammonium sulfate solution and the adsorption system;
a volume space velocity at which the ammonium sulfate solution flows through the adsorption system is less than or equal to 4 h⁻¹.

11. The method for reactivating alkanolamine solution according to claim 3, wherein a volume V₁ of the ammonium sulfate solution, a volume molar concentration C₁ of the ammonium sulfate solution, a volume V₀ of the to-be-reactivated alkanolamine solution, and a volume molar concentration C₀ of hydrogen phosphate ions in the to-be-reactivated alkanolamine solution satisfy the following relationship:
$${V}_{1} * {C}_{1} = 1.5 {V}_{0} * {C}_{0} ; and / or ,$$
the ammonium sulfate solution is allowed to flow through the adsorption system to achieve the first mixing treatment of the ammonium sulfate solution and the adsorption system;
a volume space velocity at which the ammonium sulfate solution flows through the adsorption system is less than or equal to 4 h⁻¹.

12. The method for reactivating alkanolamine solution according to claim 4, wherein a volume V₁ of the ammonium sulfate solution, a volume molar concentration C₁ of the ammonium sulfate solution, a volume V₀ of the to-be-reactivated alkanolamine solution, and a volume molar concentration C₀ of hydrogen phosphate ions in the to-be-reactivated alkanolamine solution satisfy the following relationship:
$${V}_{1} * {C}_{1} = 1.5 {V}_{0} * {C}_{0} ; and / or ,$$
the ammonium sulfate solution is allowed to flow through the adsorption system to achieve the first mixing treatment of the ammonium sulfate solution and the adsorption system;
a volume space velocity at which the ammonium sulfate solution flows through the adsorption system is less than or equal to 4 h⁻¹.

13. The method for reactivating alkanolamine solution according to claim 1, wherein the ammonium sulfate solution has a volume molar concentration of 1.2-2.3 mol/L.

14. The method for reactivating alkanolamine solution according to claim 2, wherein the ammonium sulfate solution has a volume molar concentration of 1.2-2.3 mol/L.

15. The method for reactivating alkanolamine solution according to claim 1, wherein the method for reactivating the alkanolamine solution further comprises: performing, by using a regeneration liquid, a regeneration treatment on the to-be-regenerated remover to obtain a regenerated remover;
the regeneration liquid is selected from at least one of a sodium hydroxide aqueous solution and a potassium hydroxide aqueous solution.

16. The method for reactivating alkanolamine solution according to claim 15, wherein hydroxide ions in the regeneration liquid has a concentration of 1.3-1.5 mol/L.

17. The method for reactivating alkanolamine solution according to claim 16, wherein a volume V₃ of the regeneration fluid and a volume V₂ of the to-be-regenerated remover satisfy the following relationship:
V₃≥3V₂.

18. The method for reactivating alkanolamine solution according to claim 17, wherein V₃=4V₂.

19. The method for reactivating alkanolamine solution according to claim 15, comprising allowing the regeneration liquid to flow through the to-be-regenerated remover to achieve the regeneration treatment on the to-be-regenerated remover by the regeneration liquid;
a volume space velocity at which the regeneration liquid flows through the to-be-regenerated remover is greater than or equal to 4 h⁻¹.

20. The method for reactivating alkanolamine solution according to claim 18, wherein a volume space velocity at which the regeneration liquid flows through the to-be-regenerated remover is 6 h⁻¹.
